(19) United States
(12) Patent Application Publication
(10) Pub. No.: US 2026/0215738 A1
(43) Pub. Date: Jul. 30, 2026

(54) SYSTEM AND METHOD FOR PATIENT MONITORING BASED ON CONDITION AND ACTIVITY LEVEL

(71) Applicant: KONINKLIJKE PHILIPS N.V., EINDHOVEN (NL)

(72) Inventors: WARNER RUDOLPH THEOPHILE TEN KATE, WAALRE (NL); ALBERTO GIOVANNI BONOMI, EINDHOVEN (NL); GABRIELE PAPINI, EINDHOVEN (NL); LIEKE GERTRUDA ELISABETH COX, EINDHOVEN (NL)

(21) Appl. No.: 19/146,975

(22) PCT Filed: Dec. 28, 2023
(86) PCT No.: PCT/EP2023/087875

§ 371 Date: Jul. 10, 2025

Related U.S. Application Data
(60) Provisional application No. 63/438,088, filed on Jan. 10, 2023.

(51) Int. Cl. A61B 5/00; A61B 5/11; G16H 40/67; G16H 50/30

(52) U.S. Cl. CPC A61B 5/7285; A61B 5/1115; A61B 5/1116; A61B 5/1118; G16H 40/67; G16H 50/30

(57) ABSTRACT

A patient monitoring system (400) and method (100) for determining an optimal monitoring transition of a patient (408) are provided. Sensors (402, 418) are used to measure vital signs of the patient according to a monitoring schedule and to generate sensor data. A patient monitor (404) is configured to receive the sensor data from the sensors to send to a processor. An activity monitoring apparatus (406) generates patient activity metrics, which are used by the processor along with the sensor data to generate a recommendation to alter the monitoring schedule and/or to switch measuring the vital signs of the patient via at least one second sensor.

### FIELD OF THE DISCLOSURE

The present disclosure relates generally to patient monitoring, and more specifically to systems and methods for adaptive patient monitoring.

### BACKGROUND

Patient monitoring is the healthcare field an important tool for making decisions related to treating patients effectively and efficiently, and for providing positive outcomes. For example, patient monitoring can be provided at various levels of obtrusiveness, such as spot checks by nurses or clinical staff (less obtrusive), wearable devices (minimally obtrusive), and/or telemetry devices (most obtrusive). Even with patient monitoring in place, patient information can be easily lost, missed, or otherwise overlooked if the necessary information is not collected or detected at particular times or under particular conditions. This may lead to a misdiagnosis, failed detection of a condition, and/or a premature discharge of the patient. While patients require monitoring of their vital signs and/or other parameters, it is impossible for nurses and other clinical staff to continuously monitor large volumes of patients simultaneously. Further, because patient monitoring equipment can impose a burden on the patient, intrusive patient monitoring that can provide more continuous monitoring may not be desirable in every situation.

### SUMMARY OF THE DISCLOSURE

Applicants recognized and appreciated that there exists a need to determine, without intervention from clinical staff, based on the patient condition, progress and/or activity level, an optimal point to transition a patient from bedside to ambulatory monitoring using wireless sensors and/or to generate a recommendation to alter a monitoring schedule.

According to an embodiment of the present disclosure, a patient monitoring method for determining an optimal monitoring transition of a patient is provided. The method comprises: measuring, via at least a first vital sign sensor, one or more vital signs of the patient according to a monitoring schedule and generating sensor data; communicating, to a patient monitor comprising a sensor interface configured to receive vital sign sensor data, at least the vital sign sensor data from at least the first vital sign sensor; generating, via an activity monitoring apparatus, one or more patient activity metrics measured for the patient over a period of time; generating, via at least one processor, a recommendation to alter the monitoring schedule and/or switch to measuring one or more vital signs of the patient using at least a second vital sign sensor, wherein the recommendation is generated based on at least the vital sign sensor data from at least the first vital sign sensor, the one or more patient activity metrics measured over the period of time, and a clinical information of the patient.

In an aspect, the patient monitoring method further includes: calculating a composite monitoring transition score based on at least the vital sign sensor data from at least the first vital sign sensor, the one or more patient activity metrics, and the clinical information of the patient; wherein the recommendation is generated when the composite monitoring transition score exceeds a predetermined threshold.

In an aspect, the activity monitoring apparatus includes at least one movement sensor configured to measure at least one of an activity state and a posture of the patient and to generate movement sensor data, wherein the one or more patient activity metrics comprise the movement sensor data.

In an aspect, the one or more vital signs include one or more of oxygen saturation (SpO2), respiration, heart rate, pulse rate, breathing rate, temperature, blood pressure and heart rate variability.

In an aspect, the one or more patient activity metrics includes (i) a number and duration of patient positions, posture transitions, and/or bed leaves; and (ii) a time of day occurrence of patient positions, posture transitions, and/or bed leaves.

According to another embodiment of the present disclosure, a patient monitoring system for determining an optimal monitoring transition of a patient is provided. The system includes: at least a first vital sign sensor configured to measure one or more vital signs of the patient according to a monitoring schedule and to generate vital sign sensor data; a patient monitor in communication with at least the first vital sign sensor, the patient monitor comprising a sensor interface configured to receive vital sign sensor data from one or more vital sign sensors; an activity monitoring apparatus configured to measure activity of the patient and generate patient activity metrics; and at least one processor configured to generate a recommendation to alter the monitoring schedule and/or switch to measuring one or more vital signs of the patient via at least a second vital sign sensor, wherein the recommendation is generated based on at least the vital sign sensor data from at least the first vital sign sensor, the one or more patient activity metrics measured over the period of time, and a clinical information of the patient.

In an aspect, the at least one processor is further configured to calculate a composite monitoring transition score based on at least the vital sign sensor data from at least the first vital sign sensor, the one or more patient activity metrics, and the clinical information of the patient; and wherein the recommendation to alter the monitoring schedule and/or switch to measuring one or more vital signs of the patient via at least the second vital sign sensor is generated when the composite monitoring transition score exceeds a predetermined threshold.

In an aspect, the activity monitoring apparatus includes a movement sensor configured to measure an activity state of the patient and/or a posture of the patient, wherein the one or more patient activity metrics comprise sensor data from the movement sensor.

In an aspect, the movement sensor comprises an air pressure sensor associated with the patient.

In an aspect, the activity monitoring apparatus comprises a pressure sensor associated with a patient's bed and/or a camera.

In an aspect, the one or more vital signs comprise one or more of oxygen saturation (SpO2), respiration, heart rate, pulse rate, breathing rate, temperature, blood pressure and heart rate variability.

In an aspect, the one or more patient activity metrics includes (i) a number and duration of patient positions, posture transitions, and/or bed leaves; and (ii) a time of day occurrence of patient positions, posture transitions, and/or bed leaves.

In an aspect, the monitoring schedule comprises a durational time period that is continuous or periodical.

In an aspect, the clinical information of the patient comprises one or more of the diagnostic information, imaging results, and prior medical history.

In an aspect, at least the first vital sign sensor is in wired communication with the patient monitor and the at least the second vital sign sensor is in wireless communication with the patient monitor.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart illustrating a patient monitoring method for determining an optimal monitoring transition of a patient according to aspects of the present disclosure.

FIG. 2 is a block diagram illustrating a patient monitoring system configured to determine an optimal monitoring transition of a patient according to aspects of the present disclosure.

FIG. 3 is a plot illustrating the number of bed leaves per day for a patient over a 20-day period according to aspects of the present disclosure.

FIG. 4 is a block diagram illustrating a patient monitoring apparatus having a monitoring transition module according to aspects of the present disclosure.

FIG. 5 is a block diagram illustrating a monitoring transition module according to aspects of the present disclosure.

### DETAILED DESCRIPTION OF EMBODIMENTS

The present disclosure is directed to systems and methods of patient monitoring. More specifically, the present disclosure is directed to systems and methods of determining an optimal monitoring transition of a patient, and monitoring the patient according to the optimal monitoring transition. It should be appreciated that in many cases, patients in a hospital or other clinical setting will start to move around and become more active as they recover from a condition. Before the patient begins to recover, patient monitoring via bed-side equipment may be preferred due to the large amount of information collected by the bed-side monitor. However, these bed-side monitors are typically designed for use with non-ambulatory patients and cannot be easily moved around. As such, when the patient begins to recover and leave the bed more frequently, bed-side equipment can become burdensome to the patient and negatively impact the overall quality of patient monitoring. In contrast, patient monitors and vital sign sensors designed for ambulatory patients tend to be less obtrusive but collect less information. Accordingly, the systems and methods of patient monitoring described herein are directed to addressing the need for effectively transitioning a patient between various levels of patient monitoring in order to minimize burden to the patient. The systems and methods of patient monitoring described herein also improve patient flow and clinical staff workloads by adapting patient monitoring schedules by ensuring proper levels of patient monitoring.

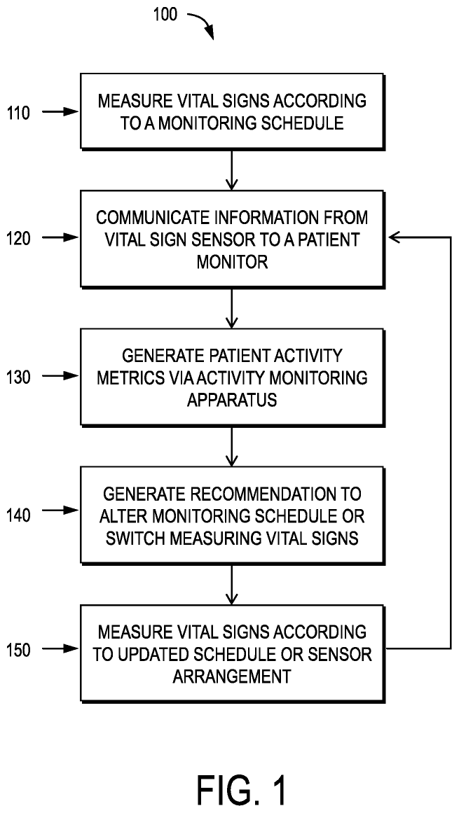

With reference to FIG. 1, a patient monitoring method 100 for determining an optimal monitoring transition of a patient is illustrated according to aspects of the present disclosure. As shown, the patient monitoring method 100 comprises: in a step 110, measuring one or more vital signs of the patient and generating vital sign sensor data, via at least a first vital sign sensor; in a step 120, communicating patient information from at least the first vital sign sensor to a patient monitor comprising a sensor interface configured to receive the vital sign sensor data; in a step 130, generating one or more patient activity metrics via an activity monitoring apparatus; and in a step 140, generating a transition recommendation to alter the monitoring schedule and/or to switch measuring the one or more vital signs of the patient via at least a second vital sign sensor, wherein the transition recommendation is generated based on at least the vital sign sensor data, the patient activity metrics measured over a period of time, and/or clinical information of the patient. In embodiments, the patient monitoring method 100 can further include: in a step 150, measuring one or more vital signs of the patient and generating vital sign sensor data via at least a second vital sign sensor.

More specifically, in the step 110, the patient monitoring method 100 includes measuring one or more vital signs of a patient and generating vital sign sensor data using at least a first vital sign sensor. In embodiments, the one or more vital signs are measurements of a physiological parameter of the patient, including but not limited to, oxygen saturation (SpO₂), respiration rate, heart rate, body temperature, invasive and/or non-invasive blood pressure, cardiac output, electrical activity associated with the heart (e.g., electrocardiogram), electrical activity associated with the brain (e.g., electroencephalogram), exhaled carbon dioxide (CO₂), and/or the like. In embodiments, the at least the first vital sign sensor may be a first set of vital sign sensors comprising two or more vital sign sensors configured to measure one or more vital signs of the patient. In some embodiments, one or more of the first set of vital sign sensors may be used to measure a common vital sign of the patient (e.g., in the case of a 12-lead ECG). In further embodiments, two or more of the first set of vital sign sensors may be used to concurrently measure different vital signs of the patient (e.g., heart rate and respiration rate, etc.).

In some embodiments, the step 110 includes measuring the one or more vital signs of the patient and generating vital sign sensor data according to a monitoring schedule. For example, a recovery protocol for a known condition may require monitoring of a specific set of parameters (e.g., vital signs) over a particular period of time and/or at predetermined time intervals. In embodiments, the monitoring schedule can be established by a governmental or regulatory agency, a hospital or network of hospitals, a care team or healthcare provider, and/or the like.

In some embodiments, the monitoring schedule, including frequency of data collection from patient monitoring devices and selection of vital signs to be monitored can be optimized based on the teachings of U.S. Pat. Nos. 10,039,451; 10,456,089; and 10,505,910 incorporated herein by reference.

In particular embodiments, at least the first vital sign sensor can include a wired vital sign sensor and/or a wireless vital sign sensor. In embodiments, at least the first vital sign sensor may further be a wearable device that is configured to be worn by the patient. In some embodiments, at least the first vital sign sensor includes a vital sign sensor that is configured to measure a vital sign of the patient in a non-ambulatory state. In embodiments, at least the first vital sign sensor can be attached or otherwise in communication with a stationary patient monitor located proximate to the patient. In other embodiments, at least the first vital sign sensor includes a vital sign sensor that is configured to measure a vital sign of the patient in an ambulatory state. In embodiments, at least the first vital sign sensor is in wireless communication with a patient monitor.

In the step 120, the patient monitoring method 100 includes communicating vital sign sensor data from at least the first vital sign sensor to a patient monitor. As mentioned above, the patient monitor can be proximate to the patient, such as in the case of a bed-side type of monitoring system that is installed with the patient's bed. Alternatively, the patient monitor can be remote from the patient and/or the first vital sign sensor, such as in the case of a nurse's station patient monitor. In embodiments, the patient monitor can be configured to capture the patient's vital signs and other parameters of relevance for the treatment of a disease or condition, to display such measurements on a screen of the patient monitor, to provide audible and/or visual alerts, and to store the patient's information with the patient's records (e.g., in a centralized electronic medical records system). In embodiments, the patient monitor can also include one or more computer processors configured convert the vital sign sensor data to suitable display information, including the possible extraction of key parameters or combining of different signals into some form of a summary trace.

In the step 130, the patient monitoring method 100 includes generating one or more patient activity metrics via an activity monitoring apparatus. In embodiments, the activity monitoring apparatus is separate from the patient monitor and is configured to monitor the physical movements of the patient within a given environment. In some embodiments, the activity monitoring apparatus can comprise one or more activity sensors, including but not limited to, motion sensors, pressure sensors, sound sensors, accelerometer sensors, gyroscope sensors, load cell sensors, optical sensors, and/or the like.

In particular embodiments, the activity monitoring apparatus can be configured to measure one or a combination of patient activity metrics using the one or more activity sensors, which can be analyzed to determine the movement/motion of the patient within the monitored environment. For example, in some embodiments, the patient activity metrics can indicate the physical location of the patient within the monitored environment, the orientation and/or posture (e.g., sitting, laying down, etc.) of the patient within the monitored environment, the activity of the patient (e.g., sleeping, etc.), the number of times the patient has left their bed, the number of times the patient has stood-up from a sitting and/or laying position, and/or the like. In further embodiments, the patent activity metrics can indicate amount of time spent in each of these positions, transitioning between positions, speed of movement of the patient, distance moved by the patient, and/or the like.

In specific embodiments, the activity monitoring apparatus may employ a camera-based system configured to capture images and/or video of an environment of the patient and detect their movement and/or positioning. One example of such camera-based system is disclosed in U.S. Pat. No. 10,223,890, incorporated herein by reference.

In further embodiments, the activity monitoring apparatus can comprise a pressure sensor and/or load cell sensor incorporated into a floormat, the patient's bed, a gurney and/or other hospital equipment supporting the patient while being monitored and treated, and configured to detect the physical presence of the patient on the bed, floormat, gurney and/or other equipment. One example of such pressure-sensor-based system is disclosed in U.S. Pat. No. 9,179,863 and U.S. Patent Publication No. 2012/0056747, both incorporated herein by reference.

In still further embodiments, the one or more activity sensors of the activity monitoring apparatus include one or more wearable sensors configured to be worn on the body of the patient. For example, in some of these embodiments, the activity monitoring apparatus is configured to determine when the patient has got out of a bed, and includes a processor for receiving measurements of the acceleration in three dimensions acting on a device that is attached to the user; and processing the measurements to determine if the patient has got out of bed, as described in detail in U.S. Patent Publication No. 2014/0313030, incorporated herein by reference.

In the step 140, the patient monitoring method 100 includes generating a recommendation to revise the monitoring protocol as it relates to the patient based on the vital sign sensor data generated by one or more vital sign sensors, the one or more patient activity metrics, and/or additional clinical information available for the patient. In embodiments, the step 140 includes generating a recommendation to alter the monitoring schedule for the patient. For example, the recommendation can include increasing or decreasing the duration and/or interval of monitoring, including but not limited to, spot checks, frequency of reviewing patient monitor, and/or the like.

In further embodiments, the step 140 includes generating a recommendation to switch to measuring one or more vital signs of the patient via at least a second vital sign sensor. For example, at least the second vital sign sensor can include a sensor configured to measure one or more physiological parameters of the patient, including but not limited to, oxygen saturation (SpO₂), respiration rate, heart rate, body temperature, invasive and/or non-invasive blood pressure, cardiac output, electrical activity associated with the heart (e.g., electrocardiogram), electrical activity associated with the brain (e.g., electroencephalogram), exhaled carbon dioxide (CO₂), and/or the like.

In embodiments, the at least the second vital sign sensor may be a second set of vital sign sensors comprising two or more vital sign sensors configured to measure one or more vital signs of the patient. In some embodiments, one or more of the second set of vital sign sensors may be used to measure a common vital sign of the patient (e.g., in the case of a 12-lead ECG). In further embodiments, two or more of the second set of vital sign sensors may be used to concurrently measure different vital signs of the patient (e.g., heart rate and respiration rate, etc.).

In particular embodiments, the at least the second vital sign sensor is different from the at least the first vital sign sensor used to measure one or more vital signs of the patient in step 110 of the patient monitoring method 100. Put another way, the step 140 can include generating a recommendation to use one or more vital sign sensors that are not currently being used.

In some embodiments, the second vital sign sensor can include a wired vital sign sensor and/or a wireless vital sign sensor. In embodiments, the second vital sign sensor may further be a wearable device that is configured to be worn by the patient. In some embodiments, at least the second vital sign sensor includes a vital sign sensor that is configured to measure a vital sign of the patient in a non-ambulatory state. In embodiments, at least the second vital sign sensor can be attached or otherwise in communication with a stationary patient monitor located proximate to the patient. In other embodiments, at least the second vital sign sensor includes a vital sign sensor that is configured to measure a vital sign of the patient in an ambulatory state. In embodiments, at least the second vital sign sensor is in wireless communication with a patient monitor.

Some examples of suitable wearable vital sign sensors are PHILIPS INTELLIVUE mx40, PHILIPS BIOSENSOR BX100 and PHILIPS HEALTHDOT, available from Royal Philips, N.A.

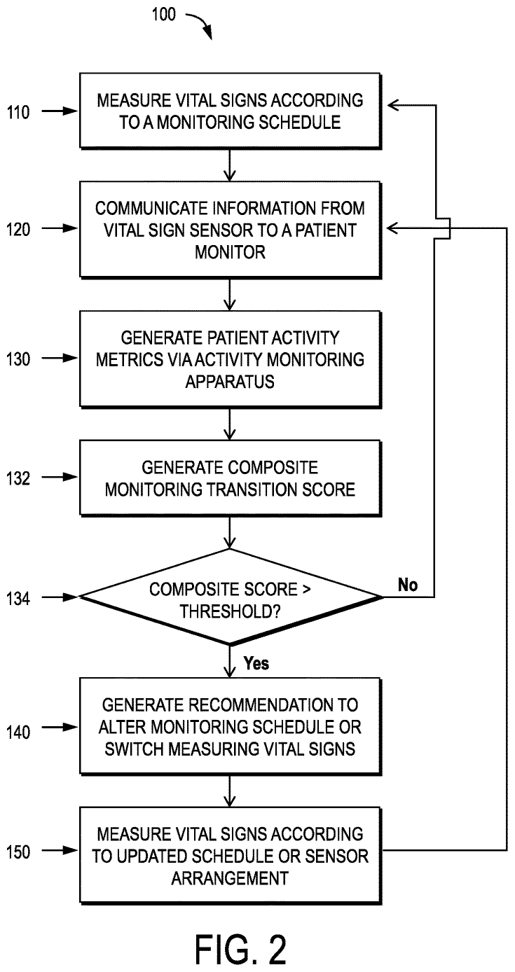

In embodiments, the step 140 of the patient monitoring method 100 can further include generating a recommendation to revise the monitoring protocol as it relates to the patient based on a composite monitoring transition score. That is, as shown in FIG. 2, the patient monitoring method 100 can further comprise: in a step 132, generating a composite monitoring transition score; in a step 134, determining whether the composite monitoring transition score exceeds a predetermined threshold. In embodiments, the composite monitoring transition score may be determined based on at least the vital sign sensor data, the patient activity metrics, and clinical information for the patient. As used herein, clinical information can include, but is not limited to, information such as identification information (e.g., date of birth, name, marital status, social security number, etc.), medical history (e.g., allergies, treatments, medical care, past and present diagnoses, habits such as diet, alcohol intake, exercise, etc.), medication information, family history, treatment history (e.g., complaints, history of illness, vital signs, physical exams, surgical history, immunization history), medical directives, lab results (e.g., lab results related to cells, tissues, or body fluids), imaging studies, consent forms, and the like.

In embodiments, the step 132 can include generating a composite monitoring score using a trained machine learning model built from a historical dataset comprising vital sign sensor data, patient activity metrics, and clinical information for a plurality of historical patients. In embodiments, the historical dataset can include an evaluation of whether a patient monitoring transition was appropriately conducted, and the machine learning model can be trained to predict whether a patient will have a successful transition. Other methods of data labeling and classification are contemplated, however, such as training the model to predict the likelihood of adverse outcomes following a monitoring transition, and/or the like.

In some embodiments, the composite monitoring transition score takes into account the patient's early warning score calculated via a cardiovascular early warning scoring (cEWS) method. The cEWS method includes the operations of: classifying the human subject using a plurality of cardiovascular deterioration classifiers each trained to classify the human subject respective to a different type of cardiovascular deterioration to generate cardiovascular early warning scores for the different types of cardiovascular deterioration, the plurality of cardiovascular deterioration classifiers operating on a set of inputs characterizing the human subject including the at least one cardiovascular parameter and the at least one respiratory parameter read by the plurality of sensors; The set of inputs may include the at least one cardiovascular parameter read by electrocardiograph electrodes and the at least one respiratory parameter comprising tidal volume read by an airflow sensor.

In other embodiments, the composite monitoring transition score takes into account the patient's early warning score calculated via a myocardial ischemia early warning method as follows. Vital sign data for a human subject are acquired using the plurality of sensors. The human subject is classified to generate an empirical myocardial ischemia score using an empirical myocardial ischemia classifier trained on a labeled data set representing training subjects with each training subject i represented by a vector Z, of features of the training subject i and a label yt representing a state of myocardial ischemia in the training subject i. The classifying includes inputting a vector to the empirical myocardial ischemia classifier that includes features generated from the acquired vital sign data for the human subject. At least one additional myocardial ischemia score is generated by applying a set of rules or a physiological model to a set of inputs characterizing the human subject including inputs generated from the acquired vital sign data for the human subject. A combined myocardial ischemia score is generated comprising a weighted combination of the empirical myocardial ischemia score and the at least one additional myocardial ischemia score.

As shown in FIG. 2, if the composite monitoring transition score is greater than a defined threshold (or falls within a predetermined range), then the patient monitoring method 100 can include generating a recommendation to change the patient's monitoring protocol in the step 140, as described herein. Otherwise, the patient monitoring method 100 can include repeating steps 110-134 until the composite score exceeds the threshold. That is, in such embodiments, the method 100 can include continuing to monitor the patient until the threshold is reached. In embodiments, the threshold may be established by the hospital and/or a care provider, or may be established through a machine learning technique.

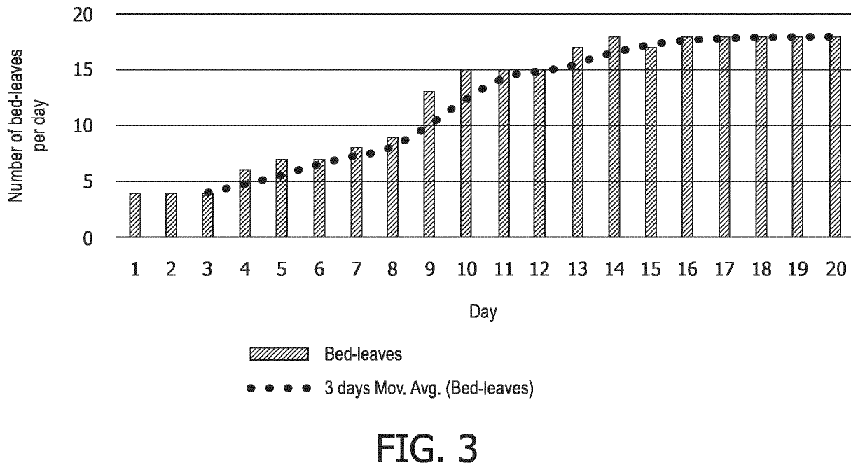

For example, with reference to FIG. 3, the patient activity metrics may be analyzed in the step 132 by image analysis, pattern recognition, and/or the like depending on the type(s) of sensors in use, in order to determine the time points of bed leaves, the duration of the leaves, and the density of bed leaves (i.e., the number of bed leaves per hour or per day). In some embodiments, these data features may be used by a trained model to determine a composite monitoring transition score. In further embodiments, additional feature data could include the time taken in leaving the bed each time and/or measurement data from other sensors. In embodiments, a time series of these values may be determined, as shown in FIG. 3, which illustrated the number of bed leaves per day for a patient and a 3-day moving average of these values. Whether these data features surpass some threshold may be determined based on a predetermined rule. For example, one example of a rule-based threshold test would be “when the total duration of bed leaves during day time exceeds 1 hour, raise a flag.” In other embodiments, the threshold determination may be based on a machine learning approach where the pattern of sensor data is classified in the intended alert classes. These alert classes may be binary recommendations (e.g., switch to an alternative monitoring arrangement vs. do not switch to an alternative monitoring arrangement), or may be on a spectrum of recommendations (e.g., the patient is frequently leaving the bed, consider switching to an alternative monitoring arrangement). In such embodiments, it should be appreciated that the machine learning algorithm can assign every output class a probability and the output class with the highest score can be taken as the current state and most desirable recommendation.

Although only the number of bed leaves are shown in FIG. 3, it should be appreciated that the composite monitoring transition score can account for additional information as described herein, including but not limited to, available vital sign sensor data. It is also contemplated that the recommendation to transition to an alternative monitoring arrangement may depend on the need to monitor certain parameters and the availability of alternative sensor devices to monitor these parameters. For example, if it is desirable to continue monitoring the patient's EEG and only the current monitoring arrangement allows for monitoring of the patient's EEG, then transitioning to an alternative monitoring arrangement might not be recommended.

Additionally, according to aspects of the present disclosure, the threshold used in the step 134 may be adjusted in response to other factors, including hospital staffing, time of day, and/or other vital signs. For example, in hospital settings, early warning scores are often used for monitored patients, where a lower average warning score per day may lead to a lower threshold on the bed leaves (or time of bed leaves, etc.) for shifting toward mobile monitoring. In contrast, if a hospital has a higher average early warning score across a care unit, the threshold for implementing an alternative monitoring arrangement for a particular patient may be raised. In some embodiments, the results of an early-warning scoring profile could also be combined with specific clinical needs to provide a recommendation to use a particular type of monitoring arrangement, such as a continuous monitoring solution like a wearable system or a more comprehensive monitoring option like a telemetry system. For example, a threshold based on bed leave events could be adjusted depending on whether the patient is at risk for arrhythmia, pneumonia, and/or another critical condition.

Returning to FIGS. 1 and 2, the patient monitoring method 100 can further include, in a step 150, measuring one or more vital signs of the patient and generating vital sign sensor data according to an alternative monitoring arrangement. As described above, the alternative monitoring arrangement can include, for example, a different monitoring schedule than the monitoring schedule that was previously implemented, and/or using a different vital sign sensor or types of vital sign sensors. In particular embodiments, the alternative monitoring arrangement can include increasing or decreasing the duration, interval, and/or method of monitoring, including but not limited to, spot checks, frequency of reviewing patient monitor, and/or the like. In further embodiments, at least a second vital sign sensor or set of vital sign sensors that are different from the first vital sign sensor or set of vital sign sensors may be used, as described above. As shown in FIGS. 1 and 2, the patient monitoring method 100 can include repeating one or more of the steps 120-150 using the alternative monitoring arrangement.

Also provided herein are patient monitoring systems adapted for determining an optimal monitoring transition of a patient. For example, the patient monitoring systems of the present disclosure may comprise: at least a first vital sign sensor configured to measure one or more vital signs of the patient according to a monitoring schedule and to generate vital sign sensor data; a patient monitor having a sensor interface configured to receive vital sign sensor data from at least the first vital sign sensor; an activity monitoring apparatus configured to generate patient activity metrics; and one or more processors configured to perform one or more steps of the methods (e.g., method 100) described above. In particular embodiments, the one or more processors can be configured to generate a recommendation to alter the monitoring schedule and/or switch to measuring the one or more vital signs of the patient via at least a second vital sign sensor.

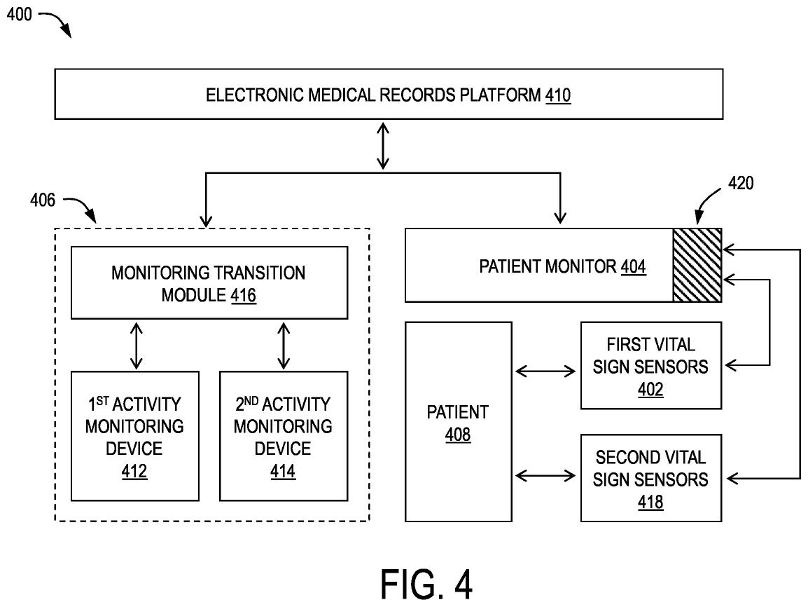

With reference to FIG. 4, one such patient monitoring system 400 is illustrated in accordance with various aspects of the present disclosure. As shown, the patient monitoring system 400 comprises at least a first vital sign sensor 402, a patient monitor 404 in communication with the first vital sign sensor 402, and an activity monitoring apparatus 406. In embodiments, the patient monitoring system 400 further comprises at least a second vital sign sensor 418.

In embodiments, the first and second vital sign sensors 402, 418 can include a first and second set of vital sign sensors 402, 418, each set comprising one or more vital sign sensors that are configured to measure one or more different vital signs. For example, in embodiments, the one or more vital signs are measurements of a physiological parameter of the patient, including but not limited to, oxygen saturation (SpO₂), respiration rate, heart rate, body temperature, invasive and/or non-invasive blood pressure, cardiac output, electrical activity associated with the heart (e.g., electrocardiogram), electrical activity associated with the brain (e.g., electroencephalogram), exhaled carbon dioxide (CO₂), and/or the like. In some embodiments, one or more of the first set of vital sign sensors 402 may be used to measure a common vital sign of the patient (e.g., in the case of a 12-lead ECG). In further embodiments, the at least the first vital sign sensor 402 may be used to concurrently measure different vital signs of the patient (e.g., heart rate and respiration rate, etc.).

In particular embodiments, one or more of the vital sign sensors 402, 418 can include a wired vital sign sensor and/or a wireless vital sign sensor. In embodiments, one or more of the vital sign sensors 402, 418 may further be a wearable device that is configured to be worn by the patient. In some embodiments, one or more of the vital sign sensors 402, 418 include a vital sign sensor that is configured to measure a vital sign of the patient in a non-ambulatory state. In embodiments, one or more of the vital sign sensors 402, 418 can be attached or otherwise in communication with a stationary patient monitor 404 located proximate to a patient 408. In other embodiments, one or more of the vital sign sensors 402, 418 can include a vital sign sensor that is configured to measure a vital sign of the patient in an ambulatory state. In embodiments, one or more of the vital sign sensors 402, 418 may be in wireless communication with a patient monitor 404.

More particularly, the patient monitor 404 can be configured to receive vital sign sensor data from the vital sign sensors 402, 418. As mentioned above, the patient monitor 404 can be proximate to a patient 408, such as in the case of a bed-side type of monitoring system 404 that is installed proximate to the patient's bed. Alternatively, the patient monitor 404 can be remote from the patient 408 and/or the vital sign sensors 402, 418, such as in the case of a nurse's station patient monitor 404. In embodiments, the patient monitor 404 can be configured to capture the patient's vital signs and other parameters of relevance for the treatment of a disease or condition, to display such measurements on a screen of the patient monitor 404, to provide audible and/or visual alerts, and to store the patient's information with the patient's records (e.g., in a centralized electronic medical records system 410). In embodiments, the patient monitor 404 can also include one or more computer processors configured convert the vital sign sensor data to suitable display information, including the possible extraction of key parameters or combining of different signals into some form of a summary trace.

In embodiments, the patient monitoring system 400 comprises an activity monitoring apparatus 406 configured to measure, extract, and/or otherwise generate one or more patient activity metrics. As shown in FIG. 4, the activity monitoring apparatus 406 is separate from the patient monitor 404 and is configured to monitor the physical movements of the patient 408 within a given environment. In some embodiments, the activity monitoring apparatus 406 can comprise one or more activity monitoring devices 412, 414, including but not limited to, motion sensors, pressure sensors, sound sensors, accelerometer sensors, gyroscope sensors, load cell sensors, optical sensors, and/or the like.

As discussed above, the activity monitoring apparatus 406 can be configured to measure one or a combination of patient activity metrics using the one or more activity monitoring device 412, 414, which can be analyzed to determine the movement/motion of a patient 408 within the monitored environment. For example, in some embodiments, the patient activity metrics can indicate the physical location of the patient 408 within the monitored environment, the orientation and/or posture (e.g., sitting, laying down, etc.) of the patient 408 within the monitored environment, the activity (e.g., sleeping, watching TV, etc.) of the patient 408, the number of times the patient 408 has left their bed, the number of times the patient 408 has stood-up from a sitting and/or laying position, and/or the like. In further embodiments, the patent activity metrics can indicate amount of time spent in each of these positions, transitioning between positions, speed of movement of the patient 408, distance moved by the patient 408, and/or the like.

In specific embodiments, the activity monitoring apparatus 406 can comprise a camera configured to record images and/or video of an environment of the patient 408. In further embodiments, the activity monitoring apparatus 406 can comprise a pressure sensor and/or load cell sensor incorporated into a floormat, the patient's bed, and/or other furniture and configured to detect the physical presence of the patient 408 on the bed, floormat, and/or other furniture. In still further embodiments, the one or more activity sensors of the activity monitoring apparatus 406 include one or more wearable sensors configured to be worn on the body of the patient 408.

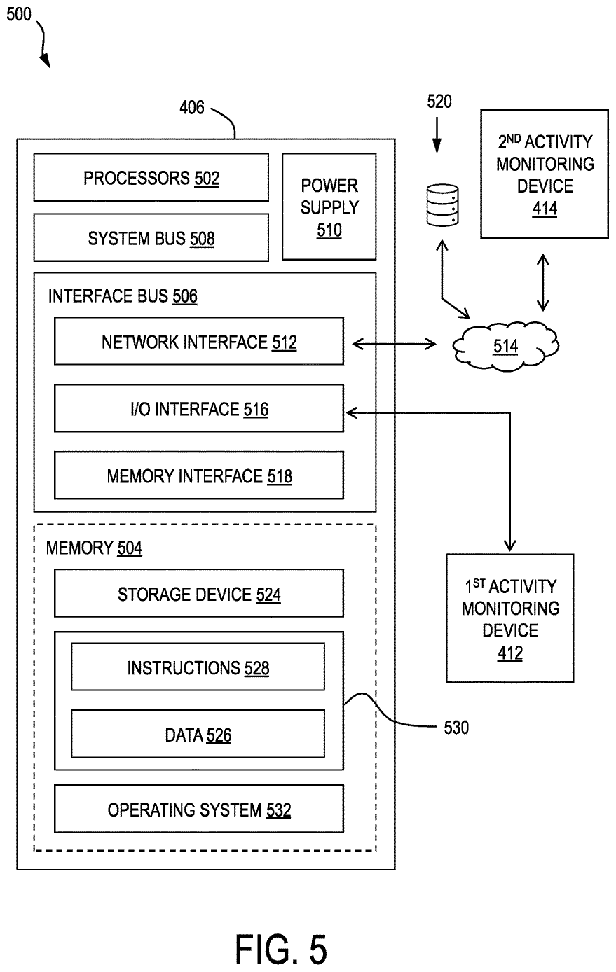

In embodiments, the patient monitoring system 400 further comprises one or more processors configured to perform one or more steps of the methods (e.g., method 100) described herein. As shown in FIGS. 4 and 5, the one or more processors may be incorporated into a monitoring transition module 416 of the activity monitoring apparatus 406. However, it is contemplated that the one or more processors may be incorporated into the patient monitor 404 and/or into a backend device such as the electronic medical records platform 410.

With reference to FIG. 5, a block diagram of a monitoring transition module 416 that may form a part of the activity monitoring apparatus 406 and/or the patient monitoring system 400 in general, is illustrated according to aspects of the present disclosure. The monitoring transition module 416 can include one or more processors 502, machine-readable memory 504, and an interface bus 506, all of which may be interconnected and/or communicate through a system bus 508 containing conductive circuit pathways through which instructions (e.g., machine-readable signals) may travel to effectuate communication, tasks, storage, and the like. The monitoring transition module 416 may be connected to a power source 510, which can include an internal power supply and/or an external power supply.

The one or more processors 502 may include a high-speed data processor adequate to execute the program components described herein and/or various specialized processing units as may be known in the art. In some examples, the one or more processors 502 may be a single processor, multiple processors, or multiple processor cores on a single die.

In some examples, the interface bus 506 may include a network interface 512 configured to connect the monitoring transition module 416 to a communications network 514, an input/output (“I/O”) interface 516 configured to connect and communicate with one or more peripheral devices 208, and/or a memory interface 518 configured to accept, communication, and/or connect to a number of machine-readable memory devices (e.g., memory 504).

The I/O interface 516 may operatively connect the monitoring transition module 416 with one or more devices, including but not limited to external, peripheral, and/or swappable devices. In some examples, these devices may include, but are not limited to, the at least the first monitoring device 412.

The network interface 512 may operatively connect the monitoring transition module 416 to a communications network 514, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In some examples, monitoring transition module 416 may communicate with one or more remote/cloud-based servers 520 (e.g., the EMR platform 410, etc.), and/or wireless devices (e.g., one or more other monitoring devices 414) via the communications network 514 and the network interface 512.

The memory 504 can be variously embodied in one or more forms of machine-accessible and machine-readable memory. In some examples, the memory 504 includes a storage device 524 comprises one or more types of memory. For example, the storage device 524 can include, but is not limited to, a non-transitory storage medium, a magnetic disk storage, an optical disk storage, an array of storage devices, a solid-state memory device, and the like, including combinations thereof.

Generally, the memory 504 is configured to store data/information 526 and instructions 528 that, when executed by the one or more processors 502, causes the monitoring transition module 416 to perform one or more tasks. In particular examples, the memory 504 includes a patient monitoring transition package 530 that comprises a collection of program components, database components, and/or data. Depending on the particular implementation, the patient monitoring transition package 530 may include software components, hardware components, and/or some combination of both hardware and software components.

The patient monitoring transition package 530 may include, but is not limited to, instructions 528 having one or more software packages configured to perform one or more of the steps of the methods (e.g., method 100) described herein. These software packages may be incorporated into, loaded from, loaded onto, or otherwise operatively available to and from the monitoring transition module 416.

In some examples, the patient monitoring transition package 530 and/or one or more individual software packages may be stored in a local storage device 524. In other examples, the patient monitoring transition package 530 and/or one or more individual software packages may be loaded onto and/or updated from a remote server 520 via the communications network 514.

In particular embodiments, the patient monitoring transition package 530 can include, but is not limited to, instructions 528 having one or more components configured to perform or facilitate the performance of the methods (e.g., method 100) described herein. These components may be incorporated into, loaded from, loaded onto, or otherwise operatively available to and from the monitoring transition module 416. In embodiments, one or more of these components can a stored program component that is executed by at least one processor, such as the one or more processors 502 of the monitoring transition module 416. In particular, the components can be configured to: (i) generate patient activity metrics; (ii) generate a recommendation to alter the monitoring schedule and/or switch to measuring the one or more vital signs of a patient 408 via at least a second vital sign sensor 418 as described above; and/or (iii) determine a composite monitoring transition score as described above.

In embodiments, the monitoring transition module 416 may also include an operating system component 532, which may be stored in the memory 504. The operating system component 532 may be an executable program facilitating the operation of the monitoring transition module 416. Typically, the operating system component 532 can facilitate access of the I/O interface, network interface, and memory interface, and can communicate with other components of the patient monitoring system 400.

In particular embodiments, the approach disclosed herein contemplates the use of a clinical remote monitoring system capable of utilizing streaming data from a plurality of connected medical devices, aggregating patient data, analyzing it to generate actionable insights and alerts, and sending timely notifications to the patient's caregivers so that they can intervene before deterioration progresses further. Preferably, such solution includes expanded interoperability into hospitals' existing mobile clinical communication and collaboration tools and electronic intensive care units (eICUs) and virtual care population health management systems, offering more visibility on live streaming data, waveforms, device alarms and contextual alerts. One suitable example of such system is PHILIPS CAPSULE SURVEILLANCE solution, available from Royal Philips, N.A

It should be appreciated that all combinations of the foregoing concepts and additional concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A patient monitoring method for determining an optimal monitoring transition of a patient, the patient monitoring method comprising:
measuring, via at least a first vital sign sensor, one or more vital signs of the patient according to a monitoring schedule and generating sensor data;
communicating, to a patient monitor comprising a sensor interface configured to receive vital sign sensor data, at least the vital sign sensor data from at least the first vital sign sensor;
generating, via an activity monitoring apparatus, one or more patient activity metrics measured for the patient over a period of time;
generating, via at least one processor, a recommendation to alter the monitoring schedule and/or switch to measuring one or more vital signs of the patient using at least a second vital sign sensor, wherein the recommendation is generated based on at least, (i) the vital sign sensor data from at least the first vital sign sensor, (ii) the one or more patient activity metrics measured over the period of time, and (iii) a clinical information of the patient.

2. The patient monitoring method of claim 1, further comprising:
calculating a composite monitoring transition score based on at least, (i) the vital sign sensor data from at least the first vital sign sensor, (ii) the one or more patient activity metrics, and (iii) the clinical information of the patient;
wherein the recommendation is generated when the composite monitoring transition score exceeds a predetermined threshold.

3. The patient monitoring method of claim 1, wherein the activity monitoring apparatus comprises at least one movement sensor configured to: (i) measure at least one of an activity state or a posture of the patient; and (ii) generate movement sensor data, wherein the one or more patient activity metrics comprise the movement sensor data.

4. The patient monitoring method of claim 1, wherein the one or more vital signs comprise one or more of oxygen saturation, respiration, heart rate, pulse rate, breathing rate, temperature, blood pressure, or heart rate variability.

5. The patient monitoring method of claim 1, wherein the one or more patient activity metrics comprise: (i) a number and duration of patient positions, posture transitions, and/or bed leaves; and (ii) a time of day occurrence of patient positions, posture transitions, and/or bed leaves.

6. A patient monitoring system for determining an optimal monitoring transition of a patient, the system comprising:
at least a first vital sign sensor configured to measure one or more vital signs of the patient according to a monitoring schedule and to generate vital sign sensor data;
a patient monitor in communication with at least the first vital sign sensor, the patient monitor comprising a sensor interface configured to receive vital sign sensor data from one or more vital sign sensors;
an activity monitoring apparatus configured to measure activity of the patient and generate patient activity metrics; and
at least one processor configured to generate a recommendation to alter the monitoring schedule and/or switch to measuring one or more vital signs of the patient via at least a second vital sign sensor, wherein the recommendation is generated based on at least; (i) the vital sign sensor data from at least the first vital sign sensor, (ii) the one or more patient activity metrics measured over the period of time, and (iii) a clinical information of the patient.

7. The patient monitoring system of claim 6, wherein the at least one processor is further configured to calculate a composite monitoring transition score based on at least: (i) the vital sign sensor data from at least the first vital sign sensor, (ii) the one or more patient activity metrics, and (iii) the clinical information of the patient; and
wherein the recommendation to alter the monitoring schedule and/or switch to measuring one or more vital signs of the patient via at least the second vital sign sensor is generated when the composite monitoring transition score exceeds a predetermined threshold.

8. The patient monitoring system of claim 6, wherein the activity monitoring apparatus comprises a movement sensor configured to measure an activity state of the patient and/or a posture of the patient, wherein the one or more patient activity metrics comprise sensor data from the movement sensor.

9. The patient monitoring system of claim 8, wherein the movement sensor comprises an air pressure sensor associated with the patient.

10. The patient monitoring system of claim 6, wherein the activity monitoring apparatus comprises a pressure sensor associated with a patient's bed and/or a camera.

11. The patient monitoring system of claim 6, wherein the one or more vital signs comprise one or more of oxygen saturation, respiration, heart rate, pulse rate, breathing rate, temperature, blood pressure or heart rate variability.

12. The patient monitoring system of claim 6, wherein the one or more patient activity metrics comprise: (i) a number and duration of patient positions, posture transitions, and/or bed leaves; and (ii) a time of day occurrence of patient positions, posture transitions, and/or bed leaves.

13. The patient monitoring system of claim 6, wherein the monitoring schedule comprises a durational time period that is continuous or periodical.

14. The patient monitoring system of claim 6, wherein the clinical information of the patient comprises one or more of the diagnostic information, imaging results, or prior medical history.

15. The patient monitoring system of claim 6, wherein at least the first vital sign sensor is in wired communication with the patient monitor and the at least the second vital sign sensor is in wireless communication with the patient monitor.
